(19) United States
(12) Patent Application Publication
(10) Pub. No.: US 2016/0356774 A1
(43) Pub. Date: Dec. 8, 2016

(54) Non-Invasive Biomarker of Antibody-Mediated Allograft Rejection

(71) Applicant: UNIVERSITY OF WASHINGTON - CENTER FOR COMMERCIALIZATION, Seattle, WA (US)

(72) Inventor: Behzad Najafian, Seattle, WA (US)

(21) Appl. No.: 15/117,118

(22) PCT Filed: Mar. 5, 2015
(86) PCT No.: PCT/US15/19036

§ 371 Date: Aug. 5, 2016

Related U.S. Application Data
(60) Provisional application No. 61/949,621, filed on Mar. 7, 2014.
(60) Provisional application No. 62/049,794, filed on Sep. 12, 2014.

(51) Int. Cl. G01N 33/569

(52) U.S. Cl. CPC G01N 33/56966; G01N 2333/70596; G01N 2800/52; G01N 2333/4716; G01N 2800/245

(57) ABSTRACT

Disclosed herein are methods for the early, non-invasive diagnosis of antibody-mediated allograft rejection, methods of identifying a population of allograft recipients at risk for developing antibody-mediated rejection and methods of monitoring the treatment of patients for antibody-mediated rejection.

### CROSS REFERENCE TO RELATED APPLICATIONS

The present application claims the benefit of U.S. Provisional patent applications 61/949,621 filed Mar. 7, 2014 and 62/049,794 filed Sep. 12, 2014, both of which are incorporated by reference herein in their entirety.

### FIELD

Disclosed herein are methods for the early, non-invasive detection of antibody-mediated allograft rejection, methods of identifying a population of allograft recipients at risk for developing antibody-mediated rejection and methods of monitoring the treatment of patients for antibody-mediated rejection.

### BACKGROUND

More than 28,000 patients receive organ transplants each year in the United States and the majority of patients suffer at least some organ rejection symptoms in spite of prophylactic immunosuppressive therapy.

Organ transplantation is currently the treatment of choice for many chronic diseases. Despite recent improvement in the short-term survival of allografts, reducing long-term graft failure is still a major challenge. Antibody-mediated rejection (AMR) is a major cause of long-term allograft loss. In excess of 20% of kidney transplant recipients develop donor-specific antibodies (DSA), which doubles their risk of allograft failure compared with patients without antibodies. Despite immunosuppressive therapy, 15-20% of kidney transplants are lost following AMR diagnosis.

Currently AMR diagnosis relies on allograft biopsy, in addition to determination of DSA. Biopsy is a costly and invasive procedure and faces several limitations. Relying solely on staining of biopsy tissue for complement component C4d may lead to AMR under-diagnosis. Conversely, presence of DSA in the plasma, without AMR-specific tissue injury on biopsy, may represent accommodation. Therefore, a diagnostic AMR biomarker should ideally reflect not only interaction of DSA with the allograft, but also injury resulted from this interaction which defines rejection.

### SUMMARY

Disclosed herein are methods to detect antibody-mediated allograft rejection (AMR), methods to maintain allograft function, and methods to monitor the treatment of AMR. The presently disclosed methods are suitable for use in recipients of heart allografts, kidney allografts, liver allografts, lung allografts, and pancreas allografts, or combinations thereof.

Thus, in certain embodiments, a method of detecting antibody-mediated rejection in an allograft transplant recipient is provided, the method comprising: determining the concentration of C4d+/CD144+ endothelial microparticles (EMP) in a blood sample obtained from the allograft transplant recipient after transplant; wherein if (i) the concentration of C4d+/Cd144+ EMP in the blood sample is greater than about seven standard deviations above the mean of a reference C4d+/CD144+ EMP concentration from a healthy individual, the allograft transplant recipient has antibody-mediated rejection and treatment for antibody-mediated rejection should be initiated, or (ii) the concentration of C4d+/Cd144+ EMP in the blood sample is less than about seven standard deviations above the mean of a reference C4d+/CD144+ EMP concentration from a healthy individual, the allograft transplant recipient does not have antibody-mediated rejection. In certain embodiments, if the concentration of CD4+/CD144+ EMP in the blood sample is greater than about eight standard deviations, or nine standard deviations, above the mean of the reference C4d+/CD144+ EMP concentration, the allograft transplant recipient has antibody-mediated rejection and treatment for antibody-mediated rejection should be initiated. In other embodiments, if the concentration of CD4+/CD144+ EMP in the blood sample is less than about eight standard deviations, or nine standard deviations above the mean of the reference C4d+/CD144+ EMP concentration, the allograft transplant recipient does not have antibody-mediated rejection.

Also disclosed is a method of monitoring treatment for antibody-mediated rejection in an allograft transplant recipient comprising: determining the concentration of C4d+/CD144+ EMP in a blood sample obtained from the allograft transplant recipient after the initiation of treatment for AMR; wherein if (i) the concentration of C4d+/Cd144+ EMP in the blood sample is greater than about seven standard deviations above the mean of a reference C4d+/CD144+ EMP concentration from a healthy individual, the antibody-mediated rejection treatment is not effective and an alternative treatment should be initiated, or (ii) the concentration of C4d+/Cd144+ EMP in the blood sample is less than about seven standard deviations above the mean of a reference C4d+/CD144+ EMP concentration from a healthy individual, the antibody-mediated rejection treatment is effective and either should be maintained or discontinued. In other embodiments, if the concentration of CD4+/CD144+ EMP in the blood sample is greater than about eight standard deviations, or nine standard deviations, above the mean of the reference C4d+/CD144+ EMP concentration, the antibody-mediated rejection treatment should be discontinued and an alternative treatment should be initiated. In yet other embodiments, if the concentration of CD4+/CD144+ EMP in the blood sample is less than about eight standard deviations, or nine standard deviations, above the mean of the reference C4d+/CD144+ EMP concentration, the antibody-mediated rejection treatment is effective and either should be maintained or discontinued.

Also disclosed herein is a method of maintaining allograft function in an allograft recipient comprising: determining the concentration of C4d+/CD144+) EMP in a blood sample from the allograft transplant recipient after transplant and before onset of transplant rejection symptoms; wherein if (i) the concentration of C4d+/Cd144+ EMP in the blood sample is greater than about seven standard deviations above the mean of a reference C4d+/CD144+ EMP concentration from a healthy individual, the allograft recipient has antibody-mediated rejection (AMR) and AMR treatment is provided to the allograft recipient; or (ii) the concentration of C4d+/Cd144+ EMP in the blood sample is less that about seven standard deviations above the mean of a reference C4d+/CD144+ EMP concentration from a healthy individual, the allograft transplant recipient does not have AMR and no treatment is necessary. In other embodiments, if the concentration of CD4+/CD144+ EMP in the blood sample is greater than about eight standard deviations, or nine standard deviations, above the mean of the reference C4d+/CD144+ EMP concentration, the allograft recipient has antibody-mediated rejection treatment and AMR treatment is provided to the allograft recipient. In yet other embodiments, if the concentration of CD4+/CD144+ EMP in the blood sample is less than about eight standard deviations, or about nine standard deviation, above the mean of the reference C4d+/CD144+ EMP concentration, the allograft transplant recipient does not have AMR and no treatment is necessary.

In yet further embodiments, a method of identifying an transplant recipient at risk of allograft rejection is provided, wherein the transplant recipient is identified by: determining the concentration of C4d+/CD144+ EMP in a blood sample obtained from the transplant recipient after transplant and before onset of transplant rejection symptoms; wherein, the transplant recipient is at risk of allograft rejection if the concentration of C4d+/CD144+ EMP in the blood sample is greater than about seven standard deviations above the mean of a reference C4d+/CD144+ EMP concentration from a healthy individual. In other embodiments, if the concentration of CD4+/CD144+ EMP in the blood sample is greater than about eight standard deviations, or about nine standard deviations, above the mean of a reference C4d+/CD144+ EMP concentration from a healthy individual, the allograft transplant recipient is at risk of allograft rejection.

In certain embodiments, the blood sample is serum, plasma, or platelet poor plasma. In other embodiments, the allograft transplant is a kidney transplant, a heart transplant, a lung transplant, a pancreas transplant, a liver transplant, or a combination thereof.

In certain embodiments, the allograft recipient has normal serum creatinine levels or alternatively, increased serum creatinine levels.

In other embodiments, the method further comprises determining donor-specific antibodies in the blood sample from the allograft transplant recipient. In still other embodiments, the method further comprises obtaining an allograft biopsy.

In some embodiments, the method is performed weekly, monthly, every other month, quarterly, yearly, or a combination thereof, after the allograft transplant.

### BRIEF DESCRIPTION OF THE DRAWINGS

FIG. 1 depicts flow cytometry scatter plots of C4d-FITC vs. CD144-PE microparticles in plasma from two kidney transplant patients, one with antibody mediated rejection (AMR; FIG. 1A) and another with no antibody mediated rejection (NAMR; FIG. 1B), and a healthy subject (FIG. 1C). There is an increase in the number of C4d+/CD144+ endothelial microparticles (EMP) in the AMR subject (arrow, top right quadrant) compared with the NAMR or healthy subject. Scatterplots from an unstained AMR sample (FIG. 1D), and mouse (FIG. 1E) and rabbit (FIG. 1F) primary antibody isotype controls stained with anti-CD144-PE and anti-C4d-FITC antibodies, respectively are shown in the bottom row.

FIG. 2 depicts a comparison of total (FIG. 2A), C4d+/CD144+ (FIG. 2B), C4d+ (FIG. 2C), and CD144+ (FIG. 2D) plasma EMP in AMR (black dots), NAMR (grey dots), and healthy subjects (white dots) by Kruskal-Wallis ANOVA test.

FIG. 3 depicts a comparison of annexin V binding (AVB+) (FIG. 3A) and C4d+/AVB+ (FIG. 3B) plasma EMP in AMR (black dots), NAMR (grey dots) and healthy subjects (white dots). No statistically significant differences were detected among the groups by Kruskal-Wallis ANOVA test.

FIG. 4 depicts a comparison of CD144+ (FIG. 4A), C4d+ (FIG. 4B), and C4d+/CD144+ (FIG. 4C) EMP concentration in relation to presence (g>0) or absence (g0) of glomerulitis, presence (cg>0) or absence (cg0) of transplant glomerulopathy, and presence (ptc>0) or absence (ptc0) peritubular capillaritis in allograft biopsies independent of AMR by Kolmogorov-Smirnov test.

FIG. 5 depicts the reduction in C4d+/CD144+ (FIG. 5A) and CD144+ (FIG. 5B) EMP concentration following treatment for AMR. FIG. 5C depicts the improvement of estimated glomerular filtration rate (eGFR) following AMR treatment in the same subjects. FIG. 5D depicts the total median fluorescence intensity (MFI) of donor-specific antibodies (DSA) which did not change statistically significantly following AMR treatment. Each line pattern and shade represents a single subject. Paired t-test was used for comparison of pre-treatment vs. post-treatment values.

FIG. 6 depicts changes in C4d+/CD144+ EMP (solid line), serum creatinine (dashed black line) and DSA MFI (dashed gray line) levels in a kidney transplant recipient between two kidney biopsies. The patient was on maintenance immunosuppression (2) of tacrolimus, mycophenolic acid, and prednisolone, and had three courses of anti-AMR therapy, anti-thymocyte globulin (1), a combination of plasmapheresis, intravenous immunoglobulin, and three doses of methylprednisolone (3), and two doses of rituximab (4).

FIG. 7 depicts the inverse relationship between eGFR and C4d+/Cd144+ EMP (EMP+) concentration in the plasma of kidney transplant recipients.

### DETAILED DESCRIPTION

The most recognized pathogenic mechanism by which antibody-mediated rejection (AMR) causes allograft endothelial injury is through activation of classical complement pathway. Endothelial microparticles (EMP) are sub-micron (0.1-1 μm) membrane-bound microvesicles that are separated from cell surface as a result of injury. Plasma EMP increase following endothelial injury, such as in hypertension, diabetes, or preeclampsia. As a result of AMR injury, allograft endothelial cells release EMP that carry complement component 4d (C4d) as a footprint of classical complement pathway activation, and the plasma concentration of these EMP, which also exhibit the endothelial marker CD144, is a useful biomarker to detect AMR.

AMR is a major cause of long term transplant loss. About 30% of kidney transplants with preformed donor-specific antibodies (DSA) develop AMR. Currently, AMR diagnosis or detection requires allograft biopsy which is invasive and costly. In addition, development of DSA and injury due to AMR can precede clinical symptoms of graft dysfunction, and leads to performance of invasive biopsy procedures. Availability of a sensitive and specific non-invasive biomarker of AMR may lead to earlier AMR detection and treatment, and hence, improve allograft outcome.

Thus, disclosed herein are methods to detect antibody-mediated allograft rejection (AMR), methods to maintain allograft function, and methods to monitor the treatment of AMR. The presently disclosed methods are suitable for use in recipients of heart allografts, kidney allografts, liver allografts, lung allografts, and pancreas allografts, or combinations thereof.

Antibody-mediated rejection in heart allografts occurs both early and late after transplantation and is associated with risk factors such as female gender, elevated pre-transplant patient-reactive antibodies (PRAs), development of de novo DSA after transplant, positive donor-specific crossmatch, prior sensitization to OKT3, cytomegalovirus seropositivity, prior implantation of a ventricular assist device, and retransplantation. AMR generally presents with the clinical symptoms of heart failure and evidence of left ventricular dysfunction in the absence of cellular infiltrates on endomyocardial biopsy specimens. Pathologic changes in heart tissue as a result of AMR include capillary endothelial changes, macrophage and neutrophil infiltration, interstitial edema, and linear accumulation of immunoglobulins and complement, especially C4d along the capillary endothelium.

Antibody-mediated rejection in renal allografts is associated with impaired urine output, increased serum creatinine, C4d deposition in peritubular capillaries and acute tissue injury comprising acute tubular necrosis, de novo DSA, glomerulitis or capillaritis with neutrophils or mononuclear cells, capillary thrombosis, transmural arteritis, or fibrinoid necrosis of arteries.

Approximately 25% of liver transplant recipients have some degree of rejection soon after transplantation more than 80% of the transplanted organs are functional at the end of the first year. C4d deposition is seen in posttransplant liver biopsies and correlate with histopathological findings and presence of DSA.

Antibody-mediated rejection in lung transplant is characterized by de novo DSA, bronchiolitis obliterans syndrome, C4d deposition in the lung as well as tissue infiltrate, lymphocytic bronchiolitis, pulmonary capillaritis, and diffuse alveolar hemorrhage. The incidence of DSA in lung transplants is 56% and the majority of patients who develop DSA do so within 90 days of transplantation.

After pancreas transplantation, AMR is often seen and involves DSA. In the pancreas, AMR causes graft failure through acute and/or chronic immunoglobulin and complement-induced microvascular injury and remodeling that eventually leads to graft fibrosis. Exocrine abnormalities (increase in serum amylase/lipase or decrease in urine amylase levels) and increased blood sugar are signs that the pancreas allograft is not functioning properly. Approximately 75% of cases of acute AMR in pancreas allograft recipients were diagnosed in the first six months posttransplantation, later occurring cases are not unusual, similar to what is seen in renal allografts.

Disclosed herein is that AMR in allograft recipients was associated with not only increased EMP, but also increased C4d+/CD144+ EMP compared to healthy subjects or transplant subjects with no rejection, or non-antibody mediated rejection (NAMR). Similarly, AMR treatment was associated with significant decline in plasma concentration of C4d+/CD144+ EMP. NAMR subjects, regardless of DSA or ATCMR (acute T cell mediated rejection), have more total, C4d+, CD144+, and C4d+/CD144+ EMP in plasma compared with healthy subjects. This is suggestive of an overall increased generation or reduced clearance of EMP in kidney transplant patients.

The concentration of C4d+/CD144+ EMP in the plasma of kidney transplant recipients who underwent a for-cause allograft biopsy reliably discriminates between AMR and non-antibody mediated rejection (NAMR) with perfect sensitivity and specificity at a threshold of about 3200 μL⁻¹.

Thus an allograft recipient with a plasma concentration of C4d+/Cd144+ EMP of greater than about 3200 μL⁻¹ is determine to have AMR. In other embodiments, an allograft recipient with a plasma concentration of C4d+/CD144+ EMP of greater than about seven, about eight, or about nine standard deviations above the mean of a reference sample comprising C4d+/CD144+ EMP from healthy individuals is to determine to have AMR. For the purposes of the present disclosure the term “about” refers to a value within 10% of the indicated number. Conversely, an allograft recipient with a plasma concentration of C4d+/CD144+ EMP of less than about seven, about eight, or about nine standard deviations above the mean of a reference sample comprising C4d+/CD144+ EMP from healthy individuals, does not have AMR.

As used herein, a “heathy individual” is an individual who has not ever received a tissue or organ transplant.

As used herein the term “endothelial microparticle” or “EMP” refers a plasma membrane vesicle shed from an injured endothelial cell. The size of endothelial microparticle ranges from 0.1 μm to 1 μm in diameter. The surface markers of endothelial microparticles are the same as endothelial cells. Typically the surface markers include, but are not limited to, CD31, CD144, VE-Cadherin, and CD146. C4d is a split product of C4 which is a component of classical complement pathway. C4d covalently binds to thioester groups of intracellular and extracellular proteins. This property makes this molecule a sensitive marker of classical complement pathway activation. Therefore an exemplary endothelial microparticle, which also reflects classical complement pathway activation, is a C4d+/CD144+ microparticle.

The term “blood sample” refers to a whole blood, serum, plasma, or platelet poor plasma (PPP) sample obtained from an allograft recipient. In certain embodiments, the blood sample is a plasma sample. A plasma sample may be obtained using methods well known in the art. For example, blood may be drawn from the patient following standard venipuncture procedure in suitable buffer. PPP may then be obtained from the blood sample following standard procedures including but not limited to, centrifuging the blood sample at about 1,500×g for about 15-20 min (room temperature), followed by pipeting of the plasma layer. PPP can also be obtained following centrifugation at about 2,600×g to about 13,000×g for 5-20 min. An exemplary blood sample is a PPP sample.

Standard methods for detecting EMP are well known in the art. For example the methods may consist in collecting a sample containing EMP from a patient and using differential binding partners directed against the specific surface markers of endothelial microparticles, wherein endothelial microparticles are bound by the binding partners to the surface markers. As disclosed herein, binding partners to CD144 and C4d are used to detect C4d+/D144+ EMP. The binding partners may be antibodies, either polyclonal or monoclonal, directed against CD144 and C4d on the endothelial microparticles. In another embodiment, the binding partners may be a set of aptamers specific for CD144 and C4d.

Polyclonal antibodies, or fragments thereof, can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Antibodies useful in practicing the claimed methods can be polyclonal or monoclonal antibodies.

Monoclonal antibodies, or fragments thereof, can be prepared and isolated using any technique that provides for the production of antibody molecules by continuous cell lines in culture. Techniques for production and isolation include but are not limited to the hybridoma technique.

In other embodiments, the binding partner may be an aptamer. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consist of conformationally constrained antibody variable regions displayed by a platform protein, such as E. coli thioredoxin A, that is selected from combinatorial libraries by two hybrid methods.

The binding partners, such as antibodies or aptamers, may be labelled with a detectable molecule or substance, such as a fluorescent molecule, a radioactive molecule, or any other labels known in the art that generally provide (either directly or indirectly) a signal.

As used herein, the term “labelled”, with regard to the antibody or aptamer, is intended to encompass direct labelling of the antibody or aptamer by coupling (i.e., physically linking) a detectable substance, such as a radioactive agent or a fluorophore (e.g. fluorescein isothiocyanate (FITC), phycoerythrin (PE), phycoerythrin-cyanine 5 (PECy5) or indocyanine (Cy5)) to the antibody or aptamer, as well as indirect labelling of the aptamer or antibody by reactivity with a detectable substance. An antibody or aptamer may be labelled with a radioactive molecule by any method known in the art. For example radioactive molecules include, but are not limited to, I¹²³, I¹²⁴, I¹¹¹, Re¹⁸⁶, and Re¹⁸⁸.

In certain embodiments, the antibodies against the surface markers are directly conjugated to a fluorophore (e.g. FITC-conjugated and/or PECy5-conjugated). Non-limiting examples include anti-human C4d-FITC, anti-human CD144-PE, and anti-human anexin V-PECy5.

The aforementioned assays may involve the binding of the binding partners (i.e. antibodies or aptamers) to a solid support. Solid supports which can be used include substrates such as nitrocellulose (e.g., in membrane or microtiter well form), polyvinylchloride (e.g., sheets or microtiter wells), polystyrene latex (e.g., beads or microtiter plates), polyvinylidine fluoride, diazotized paper, nylon membranes, activated beads, magnetically responsive beads, and the like. The solid surfaces are preferably beads. Since endothelial microparticles have a diameter of roughly 0.1-1 μm, the beads should have a diameter larger than 1 μm. Beads may be made of different materials, including but not limited to glass, plastic, polystyrene, and acrylic. In addition, the beads can be fluorescently labelled.

In certain embodiments, methods of flow cytometry are used for determining the level of endothelial microparticles in the blood sample obtained from the patient. For instance, beads labelled with specific antibodies, or labelled antibodies not conjugated to beads, may be used for the determination of endothelial microparticles. For example, endothelial microparticles may be excited with 488 nm light and green and red fluorescence of FITC and PE may be measured through 530/30 nm and 585/42 nm bandpass filters, respectively. The absolute number of endothelial microparticles are then calculated.

Accordingly, in certain embodiments, the method includes obtaining a blood sample as above described; adding both labeled antibodies against surface markers that are specific to endothelial microparticles, and performing a flow cytometry on the prepared sample in order to calculate the absolute number of endothelial microparticles therein.

In additional embodiments, the methods disclosed herein further include determination of DSA and/or allograft biopsy.

In certain embodiments, as a result of the determination of the plasma concentration of C4d+/D144+ EMP being greater than about seven, about eight, or about nine standard deviations above the mean of a reference sample comprising C4d+/CD14430 EMP from healthy individuals, AMP is detected and treatment for AMP is provided to the patient under the direction of an appropriate health professional. Suitable treatments for AMP include, but are not limited to, plasmapheresis or immunoadsorption to remove circulating antibodies or immune complexes from the blood, intravenous immunoglobulin (IVIG), rituximab, bortezomib, anti-thymocyte globulin (ATG), corticosteroids (e.g., methylprednisolone), anti-CD20 antibodies (e.g., ofatumumab, ocrelizumab), anti-CD22 antibodies (e.g. epratuzumab), lymphocyte-depleting antibodies, BAFF pathway targeting agents (e.g., atacicept, belimumab), anti-CD5 antibodies (e.g. eculizumab), immunosuppressants (e.g. belatacept), or a combination thereof.

As disclosed herein, treatment for AMR in kidney allograft recipients reduces the concentration of C4d+/CD144+ EMP, confirming that these EMP are related to AMR, and that this biomarker is a valuable surveillance tool for monitoring AMR treatment.

In other embodiments, as a result of determination of the plasma concentration of C4d+/D144+ EMP being less than about seven, about eight, or about nine standard deviations above the mean of a reference sample comprising C4d+/CD144+ EMP from healthy individuals in a patient undergoing therapy for AMP, indicative of the success of the therapy, the AMR therapy is discontinued (if the therapy is no longer needed) or maintained (if the health professional believes that continued therapy will be of benefit to the patient) under the direction of the appropriate health professional.

The present disclosure provides for periodic testing of allograft recipients regardless of clinical symptoms of allograft rejection or AMR in order to provide early detection and treatment with the goal of preserving or maintaining allograft function. As used herein, the term “early” in the context of kidney transplantation refers to detection or diagnosis of AMR before the onset of clinical symptoms, including reduced glomerular filtration rate (measured by plasma clearance of iohexol, iothalamate or inulin or calculated by plasma clearance of creatinine, cystatin C or similar methods), or increased urinary total protein or albumin concentration, or urinary total protein or albumin excretion rate (calculated from 24 hour urine collected samples or on spot urine by means of calculating urine protein creatinine ratio or urine albumin creatinine ratio). Plasma samples from a patient can be obtained and tested on a weekly, monthly, every other month, quarterly, or yearly basis and the plasma concentration of C4d+/CD144+ EMP determined. For maintenance purposes, periodic measurement of C4d+/CD144+ EMP will monitor the health of the allograft and detect AMR before clinical signs become evident. For patients who have already been diagnosed with AMR, determination of C4d+/CD144+ EMP on a periodic basis will provide information on the success, or failure, of the treatment regimen, providing information for termination of treatment (if treatment is no longer needed, or is no longer effective) or altering the treatment regimen if elevated C4d+/CD144+ EMP are still present.

In other embodiments, the disclosed methods are used to determine, or confirm, the presence of AMR after the incidence of clinical symptoms.

Also disclosed herein are kits for the determination of C4d+/CD144+ EMP from plasma of allograft transplant recipients. Certain kits are a packaged combination comprising at least (i) a binding partner for detection of C4d, and (ii) a binding partner for detection of CD144. The kit can further include blood collection supplies, instructions for obtaining plasma or PPP, instructions for determination of the concentration of C4d+/CD144+ EMP in the samples, instructions for established reference values for C4d+/CD144+ EMP in healthy subjects, and instructions for interpreting the results of the determination.

In alternative embodiments, kits according to the present disclosure comprise blood collections supplies and supplies for shipping the collected blood to a central laboratory for determination of the concentration of C4d+/CD144+ EMP in the sample(s). After collecting the blood sample(s), the collection site ships the sample(s) to a central laboratory which has established reference values for C4d+/CD144+ EMP in healthy subjects.

### EXAMPLES

### Example 1

### Patient Characteristics

Study Subjects. Adult patients with kidney transplantation who underwent a for-cause allograft biopsy at the University of Washington Medical Center or University of Minnesota Medical Center, Fairview were enrolled into this study. All research protocols were approved by the Institutional Review Board (IRB) committees at the University of Washington and University of Minnesota. All subjects gave informed consent before enrollment. Patients with multi-organ transplants were excluded. Adult healthy volunteers with no known kidney disease, hypertension, or diabetes were enrolled as controls. Patients' characteristics and clinical information at biopsy were extracted from medical records (Table 1).

Fifty-nine patients with kidney transplantation at the time of for-cause biopsy and 23 healthy volunteers were enrolled (Table 1). Fifty-three (90%) transplant patients were consented prior to the biopsy. The original kidney diseases in transplant subjects are listed in Table 2. Healthy subjects were younger and included more females compared to kidney transplant patients (Table 1). Patients with AMR and no AMR (NAMR) were not different in age, gender ratios, prevalence of hypertension or diabetes, systolic or diastolic blood pressure, serum creatinine, estimated glomerular filtration rate (eGFR), or urine protein to creatinine ratio (UPCR). AMR patients had longer transplant duration (p=0.03) than NAMR subjects.

The majority of patients (76%) were receiving three drugs (a calcineurin inhibitor, prednisone, and mycophenolate mofetil (MMF) or mycophenolic acid) for maintenance immunosuppression, 14% of them were receiving two of these drugs, 6% were only on one drug (a calcineurin inhibitor or MMF), and 4% had stopped their medications prior to the biopsy.

TABLE 1
Clinical Characteristics of Study Subjects

| | AMR | | NAMR | | Healthy Subjects | |
|---|---|---|---|---|---|---|
| | N = 16 | | N = 43 | | N = 23 | |
| Male (%) | 9 | (56) | 25 | (58) | 5 | (21.7) * |
| Age (year) | 50 | [24-72] | 51 | [18-78] | 33 | [19-63] ** |

| Hypertension, N (%) | 13 | (81) | 34 | (79) | NABD |
|---|---|---|---|---|---|
| Systolic blood pressure (mmHg) | 136 ± 11 | | 133 ± 13 | | NA |
| Diastolic blood pressure (mmHg) | 79 ± 10 | | 78 ± 11 | | NA |
| Serum creatinine (mg/dL) | 2.5 ± 1.4 | | 2.1 ± 1.3 | | NA |
| eGFR (ml/min/1.73 m²) | 32 ± 14 | | 39 ± 15 | | NA |
| UPCR (mg/g) | 2.5 ± 3.9 | | 1.1 ± 1.7 | | NA |
| Diabetes, N (%) | 6 | (38) | 10 | (23) | NABD |
| Transplant vintage (months) | 57.1 | [0.3-225.3] | 5.2 | [0.0-240.2] ^{†} | NABD |
| Prior Kidney Transplant, N (%) | 4 | (25) | 4 | (9) | NABD |

* p = 0.03 vs. AMR and p = 0.005 vs. NAMR;
** p = 0.02 vs. AMR and p = 0.003 vs. NAMR;
^{†} p = 0.03 vs. AMR.
Abbreviations: AMR: antibody mediated rejection; NAMR: no antibody mediated rejection; NA: not available; NABD: not applicable by definition; eGFR: estimated GFR based on the Modification of Diet in Renal Disease (MDRD) formula; UPCR: urine protein creatinine ratio.

TABLE 2
Original Kidney Diseases in Transplant Patients by Group

| | AMR | NAMR |
|---|---|---|
| Original Kidney Disease | (N) | (N) |
| Diabetic nephropathy | 4 | 8 |
| Chronic hypertension | 1 | 4 |
| Focal segmental glomerulosclerosis | 2 | 3 |
| IgA nephropathy | 1 | 5 |
| Lupus nephritis | 1 | 2 |
| Adult polycystic kidney disease | — | 3 |
| Glomerulonephritis, not otherwise specified | 1 | 2 |
| Reflux nephropathy/obstruction | — | 3 |
| Recurrent pyelonephritis | — | 1 |
| Membranoproliferative glomerulonephritis | 1 | — |
| Henoch-Schönlein purpura | 1 | — |
| Chronic tubulointerstitial nephritis | 1 | — |
| Renal dysgenesis | 1 | — |
| Anti-GBM glomerulonephritis | — | 1 |
| C3 glomerulopathy | — | 1 |
| Unknown | 2 | 10 |
| Total | 16 | 43 |

Abbreviations: AMR = antibody-mediated rejection; NAMR = No antibody mediated rejection.

Kidney Biopsies. Allograft biopsies were processed for light and immunofluorescent microscopy using standard methods and electron microscopy when indicated. C4d staining was performed using immunofluorescence method on frozen sections. Where frozen samples were not available or adequate, the immunoperoxidase method was then used. Histological findings were scored according to Banff classification (Haas M, et al. Banff 2013 meeting report: inclusion of c4d-negative antibody-mediated rejection and antibody-associated arterial lesions. Am J Transplant. 14:272-83, 2014) by a renal pathologist prior to microparticle assays.

Sixteen patients had acute AMR, all with Banff grade 2 histologic severity. All AMR biopsies showed positive C4d staining in peritubular capillaries, except in one who had positive plasma DSA (sum MFI=6997, C1q-positive) with glomerulitis (g1) and peritubular capillaritis (ptc3), but no peritubular capillary C4d staining, consistent with C4d-negative AMR. Eleven (69%) AMR cases showed transplant glomerulopathy. None of the biopsies had pure chronic AMR (i.e. transplant glomerulopathy without microvascular inflammation). Of 16 patients with AMR, 10 (63%) had histologic evidence of concomitant acute T-cell mediated rejection (ATCMR), including Banff IIA (n=2), or borderline (n=8) ATCMR. None of the NAMR biopsies showed positive C4d staining of peritubular capillaries. Eleven (26%) of NAMR patients had evidence of ATCMR, including Banff IIA (n=1), IA (n=1) or borderline ATCMR (n=9). None of the biopsies showed de novo or recurrent glomerulonephritis, except for one NAMR biopsy with recurrent C3 glomerulopathy. Other findings in the NAMR biopsies included acute tubular injury, focal and segmental glomerulosclerosis, nonspecific chronic injury, polyomavirus nephropathy, mild glomerulitis, and no diagnostic abnormality. One NAMR case with negative DSA showed intracapillary thrombi of unknown etiology.

Blood Samples. Intravenous blood was collected from the antecubital vein into ACD-A blood collection tubes concomitant with the biopsy and processed within 1 hr while being kept at room temperature. Platelet Poor Plasma (PPP) was prepared by centrifugation at 1500×g for 20 min at 25° C. and stored at −80° C. in cryovials until flow cytometry. All blood samples were collected prior to any change in the maintenance immunosuppression and 0 (-1-16) (median (range)) days from the biopsy date. Nine paired pre- and post-AMR treatment samples were available from AMR subjects. In these cases, AMR treatment consisted of anti-thymocyte globulin (ATG) and methylprednisone (n=2); intravenous immunoglobulin (IVIG) alone (n=2); ATG, IVIG, plasmapheresis, methylprednisolone, and rituximab (n=1); ATG, IVIG, plasmapheresis and rituximab (n=1); IVIG, plasmapheresis and rituximab (n=1); IVIG and methylprednisolone (n=1); and rituximab alone (n=1). The post-treatment samples were obtained after the completion of AMR treatment which was 14 (5-27) days after the pre-treatment sample depending on the regimen used.

### Example 2

### Donor Specific Antibodies

Donors and recipients were typed for HLA using genomic DNA by the lmmunogenetics/HLA laboratories at the Puget Sound Blood Center or at the University of Minnesota Medical Center, Fairview. All transplant patients were tested for antibody to donor HLA (DSA) using a microarray bead assay (LAB Screen Singles, One Lambda, Inc., Canoga Park, Calif.). All samples were tested for IgG, those that were IgG DSA positive were assayed for C1q-binding DSA (C1q Screen, One Lambda, Inc.). IgG values were reported as median fluorescence intensity (MFI) normalized to negative controls; C1q values as exceeding a threshold of 2000 MFI.

All AMR patients had positive DSA by definition, twelve (75%) of which were C1q-positive. Five (12%) NAMR patients had positive DSA, of which 4 (9%) were C1q-positive. These NAMR subjects did not have microvascular inflammation on their biopsies to qualify for diagnosis of C4d-negative AMR. All AMR subjects had anti-HLA class II DSA and 38% of them had both anti-HLA class I and II DSA (Table 3). Eighty-nine percent of anti-HLA class II and 75% of anti-HLA class I antibodies were C1q-positive (not statistically different). Sixty-eight percent of AMR patients had anti-HLA DQ (DQ) DSA.

TABLE 3
Donor Specific Antibodies in Patients with Kidney Transplantation

| Donor Specific Antibody | AMR (N = 16) | | NAMR (N = 43) | | p-value |
|---|---|---|---|---|---|
| HLA Class I only, N (%) | 0 | (0) | 1 | (2) | NS |
| HLA Class II only, N (%) | 10 | (63) | 3 | (7) | <0.001 |
| HLA Class I and II, N (%) | 6 | (37) | 1 | (2) | 0.001 |
| HLA Class I, sum DSA MFI | 0 | [0-9809] | 0 | [0-2677] | NS |
| C1q+ HLA Class I, sum DSA MFI | 0 | [0-9809] | 0 | [0-1852] | NS |
| HLA Class II, sum DSA MFI | 17601 | [1000-36132] | 1910 | [0-5489] | <0.01 |
| C1q+ HLA Class II, sum DSA MFI | 16730 | [0-35647] | 1910 | [0-5489] | <0.01 |
| HLA Class I and II, sum DSA MFI | 19777 | [1468-44190] | 0 | [0-5489] | <0.001 |
| C1q+ HLA Class I and II, sum DSA MFI | 18674 | [0-37155] | 0 | [0-2372] | <0.001 |

Abbreviations: AMR: antibody mediated rejection; NAMR: no antibody mediated rejection; MFI: median fluorescent intensity; NS: not statistically significant.

### Example 3

### C4d+/CD144+ Plasma EMP Increased During AMR and Reliably Distinguished AMR from NAMR or Healthy Subjects

Flow Cytometry Analysis. PPP was thawed and stained with phycoerythrin (PE)-conjugated anti-human CD144 (EBioscience, clone 16B1) as an endothelial marker, and polyclonal FITC-conjugated rabbit anti-human C4d (American Research Products) and phycoerythrin-cyanine 5 (PECy5)-conjugated annexin V for testing annexin V binding (AVB). 2.5 μL of cocktails of anti-CD144 and anti-C4d, or 2.5 μL of anti-C4d and 1 μL of PECy-5-annexin V, were added to 20 μL of PPP, and incubated for 30 min at room temperature while protected from light. Samples of the cocktail containing PECy5-annexin V were further incubated for 30 min with the addition of HEPES Buffer (10 mM HEPES, 140 nM NaCl, 4.5 mM KCl, 1% bovine serum albumin, 0.1% sodium azide) and CaCl₂ (0.025 M mixed 1:3 with HEPES) for annexin V binding. An Apogee A-50 Micro (Apogee Flow Systems Ltd, Hemel Hempstead, UK) with a single 488 nm blue laser was used for flow cytometry. Antibody specific isotype controls (rabbit primary antibody isotype control (Invitrogen) for rabbit anti-human C4d-FITC; and mouse primary antibody isotype control (Invitrogen) for mouse anti-human CD144-PE) were used to determine the background noise. Initial background gating was determined by buffer only. Unstained patient samples were used to set the final gates for microparticles. Photomultiplier tube (PMT) voltages remained the same for all samples regardless of whether they were stained or unstained. Threshold values remained constant for both forward-scatter and side-scatter for all samples.

Apogee Histogram software (V120) was used for data analysis and preparing plots. Apogee mixed calibration silica beads (Apogee Flow Systems) were used to check laser functionality and to set microparticle size gate at 100 nm to 1000 nm. All analyses were performed on results obtained after size gating. Inter-assay variability (reproducibility) of the assay was examined by running five triplicate samples prepared and stained independently.

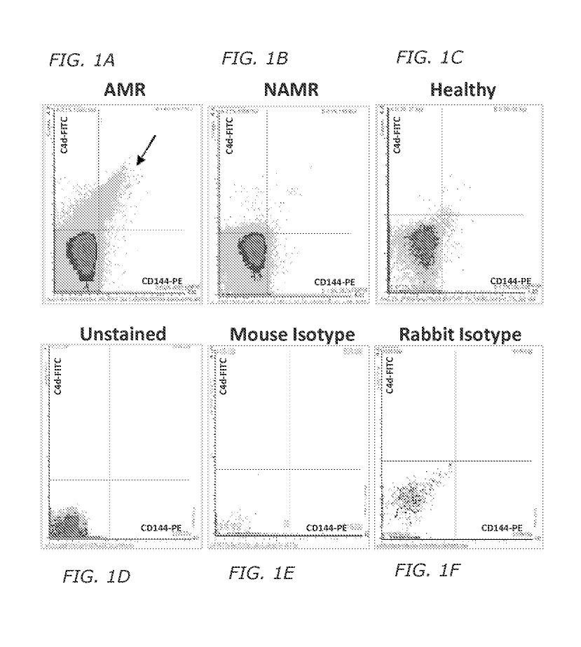

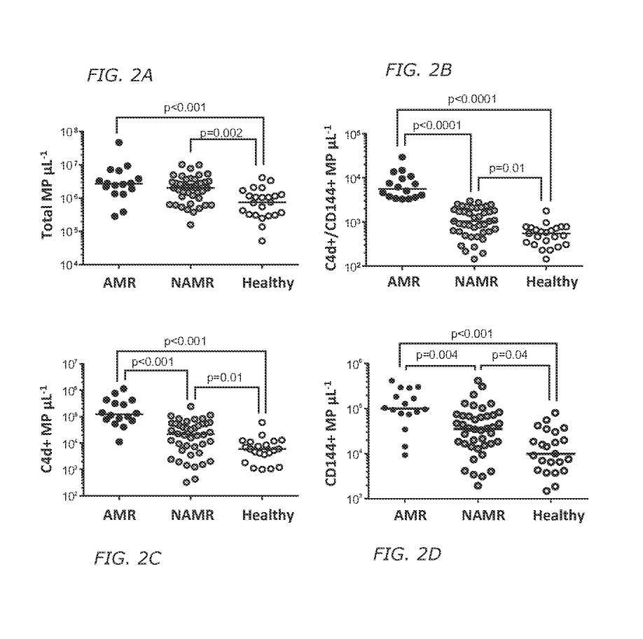

Plasma concentration of C4d+/CD144+ EMP was remarkably increased in AMR patients compared with NAMR or healthy subjects (FIG. 1) and reliably discriminated AMR patients from those without AMR (FIG. 2 and Table 5). There was no overlap between the range of C4d+/CD144+ EMP concentration in AMR with that in the NAMR or healthy subjects, providing 100% sensitivity and 100% specificity at a concentration about 3200 μL⁻¹. Thus, AMR can be detected by a concentration of C4d+/CD144+ EMP in the plasma greater than seven standard deviations above the mean of the C4d+/CD144+ EMP concentration of healthy subjects. C4d+/CD144+ EMP were also more abundant in NAMR compared to healthy subjects (p=0.01). The only C4d-negative AMR patient had 4066 C4d+/CD144+ EMP per μL.

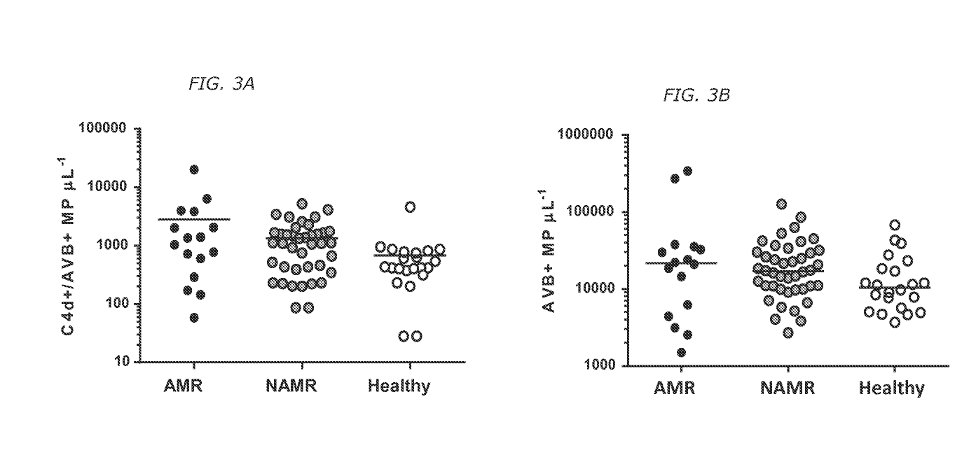

C4d+ and CD144+ EMP were also more abundant in plasma from AMR and NAMR patients compared to healthy subjects, and in AMR compared to NAMR patients. Plasma total EMP were remarkably increased in AMR and NAMR (p=0.002) compared to healthy subjects, but were not statistically different between AMR and NAMR groups (FIG. 2). A minor fraction of EMP (2 ±7%) were AVB+. While total number of AVB+EMP were not different among the groups, C4d+/AVB+ EMP concentration was more abundant in NAMR and AMR groups compared to healthy subjects, but was not different between AMR and NAMR subjects (FIG. 3 and Table 6).

TABLE 5

| | Healthy | | | | NAMR | | | | AMR | | | |
|---|---|---|---|---|---|---|---|---|---|---|---|---|
| | | | | C4d+/ | | | | C4d+/ | | | | C4d+/ |
| | Total | C4d+ | CD144+ | CD144+ | Total | C4d+ | CD144+ | CD144+ | Total | C4d+ | CD144+ | CD144+ |
| minimum | 52058 | 1002 | 1496 | 144 | 158964 | 328 | 678 | 270 | 284434 | 11084 | 9300 | 3280 |
| maximum | 4114366 | 59770 | 80600 | 1782 | 10198070 | 240892 | 418072 | 2992 | 46524020 | 1142080 | 420264 | 29468 |
| mean | 1068958 | 8936 | 18913 | 573 | 2631638 | 36142 | 60455 | 1258 | 5926430 | 260728 | 149007 | 8145 |
| SD | 1028770 | 12023 | 19920 | 343 | 2342330 | 44330 | 82063 | 804 | 11106799 | 307394 | 119529 | 6796 |

TABLE 6

| | Healthy | | NAMR | | AMR | |
|---|---|---|---|---|---|---|
| | | C4d+/ | | C4d+/ | | C4d+/ |
| | AVB+ | AVB+ | AVB+ | AVB+ | AVB+ | AVB+ |
| minimum | 3684 | 28 | 4038 | 202 | 1496 | 58 |
| maximum | 67942 | 1604 | 85324 | 4120 | 340488 | 20058 |
| mean | 16085 | 685 | 24180 | 1330 | 53954 | 2799 |
| SD | 15836 | 914 | 23529 | 1175 | 99545 | 4914 |

There was no relationship between transplant vintage and plasma concentrations of any of the studied EMP subpopulations in AMR and NAMR patients, or between age and microparticle concentrations in healthy subjects. No relationship was found between EMP concentration and blood pressure, estimated glomerular filtration rate (eGFR), urine protein creatinine ratio (UPCR), and sum or maximum DSA MFI, or C1q+ sum or maximum DSA MFI or DQ DSA MFI in AMR or NAMR patients.

### Example 4

### Relationships Between Microvascular Inflammation and C4d+/CD144+ EMP

Severity of AMR is primarily determined based on biopsy findings; however, Banff AMR grading is not sensitive and in practice, most AMR biopsies are classified as grade 2 based on presence of microvascular inflammation. Moreover, Banff scoring is subject to interobserver variability among pathologists.

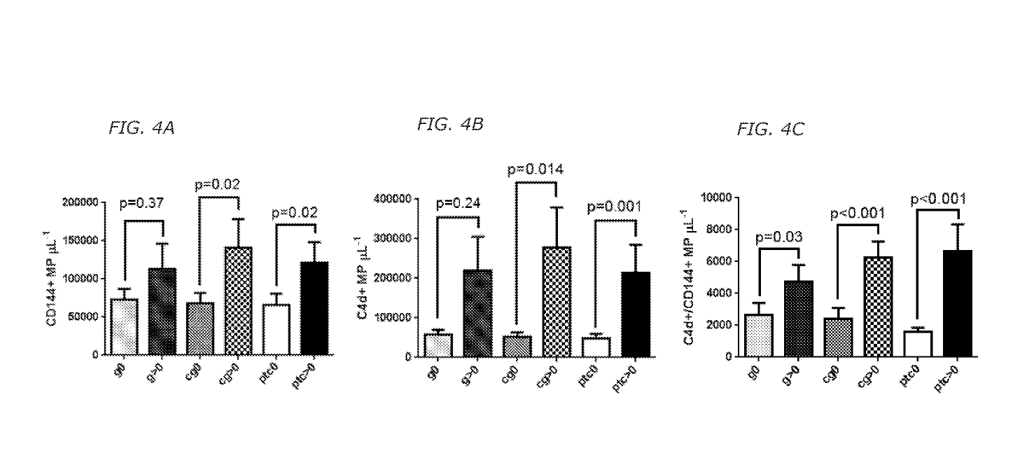

Among all transplant patients, C4d+/CD144+ plasma EMP concentration was greater in patients with glomerulitis, peritubular capillaritis or transplant glomerulopathy compared to those without these findings on allograft biopsies (FIG. 4 and Table 7). Moreover, microvascular inflammation scores g (r=0.34, p=0.02), ptc (r=0.59, p=0.0005) and ptc+g (r=0.56, p=0.0001) and transplant glomerulopathy score cg (r=0.62, p=0.00001) were directly related to C4d+/CD144+ plasma EMP concentration. However, when the analyses were limited to AMR patients, no statistically significant relationship was found between these microparticle subpopulations and severity of microvascular inflammation or transplant glomerulopathy. Similarly, no relationship was found between DSA MFI values and microvascular inflammation scores among AMR subjects.

TABLE 7

| | | | |
|---|---|---|---|
| Mean | SD | Minimum | Maximum |

C4d+/CD144+

| | | | | |
|---|---|---|---|---|
| g0 | 2658 | 4879 | 144 | 29468 |
| g > 0 | 4726 | 4130 | 216 | 13700 |

C4d+

| | | | | |
|---|---|---|---|---|
| g0 | 57708 | 76696 | 328 | 425778 |
| g > 0 | 219512 | 331919 | 432 | 1142080 |

CD144+

| | | | | |
|---|---|---|---|---|
| g0 | 72878 | 90878 | 1956 | 418072 |
| g > 0 | 113489 | 127028 | 678 | 420264 |

C4d+/CD144+

| | | | | |
|---|---|---|---|---|
| cg0 | 2389 | 4727 | 144 | 29468 |
| cg > 0 | 6250 | 3540 | 2284 | 13706 |

C4d+

| | | | | |
|---|---|---|---|---|
| cg0 | 52864 | 74598 | 328 | 425778 |
| cg > 0 | 278129 | 349978 | 11084 | 1142080 |

CD144+

| | | | | |
|---|---|---|---|---|
| cg0 | 68353 | 88616 | 678 | 418072 |
| cg > 0 | 140611 | 130647 | 3120 | 420264 |

C4d+/CD144+

| | | | | |
|---|---|---|---|---|
| ptc0 | 1602 | 1523 | 144 | 7520 |
| ptc > 0 | 6669 | 7103 | 1008 | 29468 |

C4d+

| | | | | |
|---|---|---|---|---|
| ptc0 | 47375 | 77861 | 328 | 422352 |
| ptc > 0 | 214935 | 296724 | 11084 | 1142080 |

CD144+

| | | | | |
|---|---|---|---|---|
| ptc0 | 66166 | 91890 | 678 | 418072 |
| ptc > 0 | 121262 | 114916 | 7482 | 420264 |

### Example 5

### AMR Treatment is Associated with a Decline in C4d+/CD144+ and CD144+ EMP in Plasma

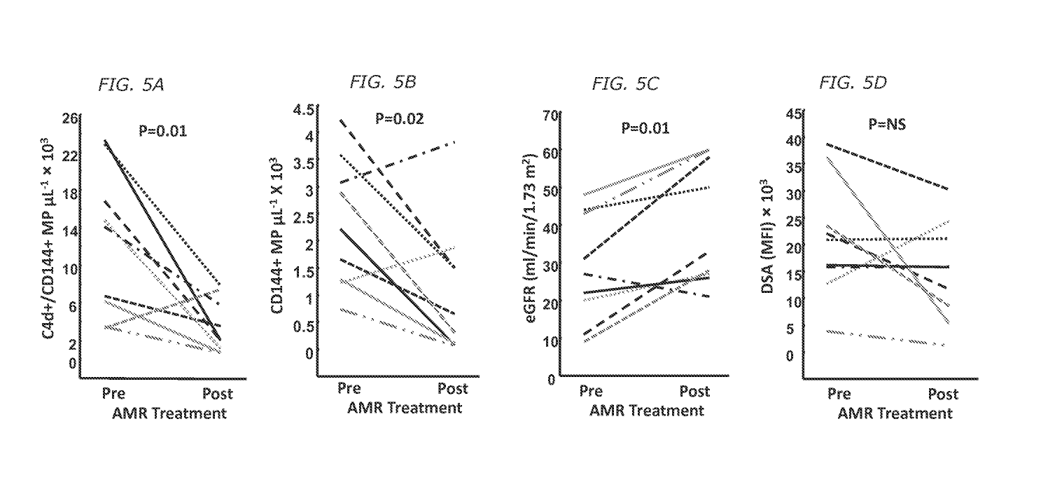

In 9 cases, where paired pre- and post-AMR treatment samples were available, treatment was associated with eGRF improvement from 28±14 to 40±16 ml/min/1.73 m² (p=0.02) and with a decline in concentration of C4d+/CD144+ and CD144+ EMP (FIG. 5 and Table 8) in the plasma. However, the change in C4d+/CD144+ or total CD144+ EMP pre- and post-treatment did not correlate with the change in eGFR. Although on average, DSA sum MFI was reduced after AMR treatment, the difference was not statistically significant. Pre- and post-treatment values for total EMP, total C4d+, total AVB+, and C4d+/AVB+ EMP were not statistically different. No follow up biopsies were available after AMR treatment in these patients.

TABLE 8

| | | | CD4+/ | | | | | |
|---|---|---|---|---|---|---|---|---|
| | CD144+ | | CD144+ | | eGFR | | DSA | |
| | Pre | Post | Pre | Post | Pre | Post | Pre | Post |
| minimum | 74016 | 6100 | 3340 | 692 | 9 | 21 | 3900 | 1200 |
| maximum | 722970 | 382010 | 23308 | 7826 | 48 | 60 | 38691 | 30228 |
| mean | 232317 | 110158 | 12403 | 3469 | 28 | 40 | 21112 | 14914 |
| SD | 119067 | 123723 | 7824 | 2843 | 14 | 16 | 10988 | 9321 |

Coefficients of variations of 5 triplicate samples were 14% for C4d+/CD144+, 19% for C4d+, 28% for CD144+ EMP, and 11% for total EMP, indicative of adequate repeatability of the assay.

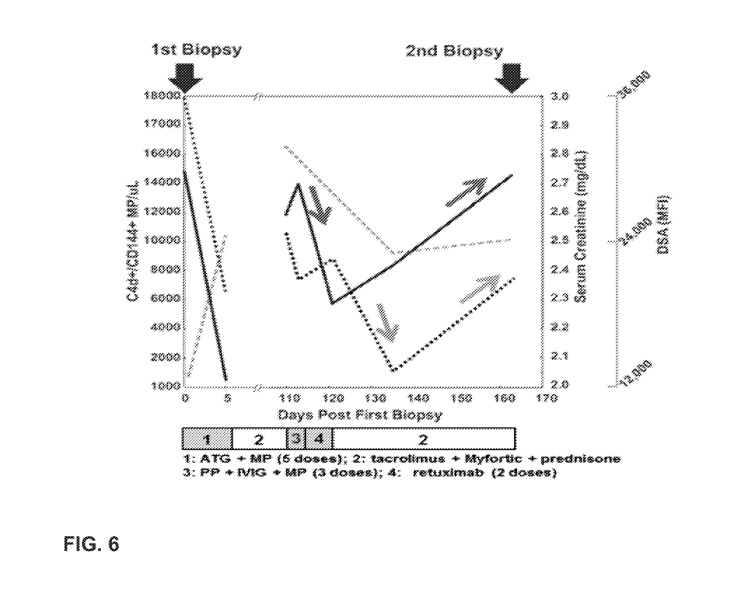

Seven longitudinal samples were collected from a kidney transplant patient within 160 days of a diagnosis of AMR based on an allograft biopsy (FIG. 6). The patient was on maintenance immunsuppresive therapy comprising prednisolone, tacrolimus, and mycophenolic acid. The first plasma sample (taken between days 0-5 after the first biopsy) showed that AMR treatment (anti-thymocyte globulin and methylprednisolone) was associated with a significant decline in both plasma C4d+/CD144+ EMP and serum creatinine. The next sample (day 110 post first biopsy) showed significant rise in C4d+/CD144+ EMP. AMR treatment (plasmapheresis, intravenous immunoglobulin, and three doses of methylprednisolone, followed by a course of rituximab) again was followed by a decline in C4d+/CD144+ EMP. However, a decline in serum creatinine occurred about 10 days later than the onset of decline in C4d+/CD144+ EMP. Stopping AMR treatment was associated with an increase in C4d+/CD144+ EMP, followed by increased serum creatinine 10-12 days later. These findings suggest that C4d+/CD144+ EMP changes may precede serum creatinine changes and clinical AMR.

### Example 6

### C4d+/CD144+ EMP Density Inversely Correlates with eGFR and Response to Therapy in Kidney Transplant Patients

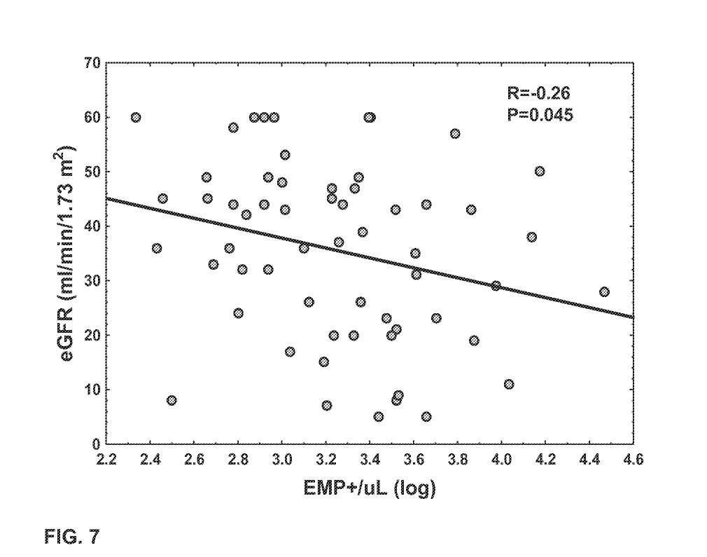

In 59 kidney transplant patients, including a mixture of patients with AMR as well as other pathologic conditions, based on allograft biopsy an inverse relationship was found between eGFR and C4d+/CD144+ EMP (FIG. 7). This finding supports that C4d+/CD144+ EMP are a prognostic biomarker for allograft survival in kidney transplant patients.

In summary, the present study introduces a highly promising, sensitive and specific noninvasive biomarker of AMR that reduces following AMR treatment. This biomarker can facilitate early detection and treatment of AMR and improve allograft survival.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method of detecting antibody-mediated rejection in an allograft transplant recipient, the method comprising:
determining the concentration of C4d+/CD144+ endothelial microparticles (EMP) in a blood sample obtained from the allograft transplant recipient after transplant;
wherein if (i) the concentration of C4d+/Cd144+ EMP in the blood sample is greater than about seven standard deviations above the mean of a reference C4d+/CD144+ EMP concentration from a healthy individual, the allograft transplant recipient has antibody-mediated rejection and treatment for antibody-mediated rejection should be initiated, or (ii) the concentration of C4d+/Cd144+ EMP in the blood sample is less than about seven standard deviations above the mean of a reference C4d+/CD144+ EMP concentration from a healthy individual, the allograft transplant recipient does not have antibody-mediated rejection.

2. The method according to claim 1, wherein the blood sample is serum, plasma, or platelet poor plasma.

3. (canceled)

4. (canceled)

5. The method according to claim 4, wherein the blood sample contains normal serum creatinine levels.

6. The method according to claim 4, wherein the blood sample contains increased serum creatinine levels.

7. The method according to claim 1, wherein the method further comprises determining donor-specific antibodies in the blood sample from the allograft transplant recipient.

8. The method according to claim 1, wherein the method further comprises obtaining an allograft biopsy.

9. (canceled)

10. The method according to claim 1, wherein if the concentration of CD4+/CD144+ EMP in the blood sample is greater than about eight standard deviations above the mean of the reference C4d+/CD144+ EMP concentration, the allograft transplant recipient has antibody-mediated rejection and treatment for antibody-mediated rejection should be initiated.

11. The method according to claim 1, wherein if concentration of CD4+/CD144+ EMP in the blood sample is less than about eight standard deviations above the mean of the reference C4d+/CD144+ EMP concentration, the allograft transplant recipient does not have antibody-mediated rejection.

12. The method according to claim 1, wherein if the allograft transplant recipient has initiated therapy for transplant rejection, the method further comprises
determining the concentration of C4d+/CD144+ EMP in a blood sample obtained from the allograft transplant recipient after the initiation of treatment for AMR;
wherein if (i) the concentration of C4d+/Cd144+ EMP in the blood sample is greater than about seven standard deviations above the mean of a reference C4d+/CD144+ EMP concentration from a healthy individual, the antibody-mediated rejection treatment is not effective and an alternative treatment should be initiated, or (ii) the concentration of C4d+/Cd144+ EMP in the blood sample is less than about seven standard deviations above the mean of a reference C4d+/CD144+ EMP concentration from a healthy individual, the antibody-mediated rejection treatment is effective and either should be maintained or discontinued.

13. (canceled)

14. (canceled)

15. (canceled)

16. (canceled)

17. (canceled)

18. (canceled)

19. (canceled)

20. (canceled)

21. A method of maintaining allograft function in an allograft recipient comprising:
determining the concentration of C4d+/CD144+ EMP in a blood sample from the allograft transplant recipient after transplant and before onset of transplant rejection symptoms;
wherein if (i) the concentration of C4d+/Cd144+ EMP in the blood sample is greater than about seven standard deviations above the mean of a reference C4d+/CD144+ EMP concentration from a healthy individual, the allograft recipient has antibody-mediated rejection (AMR) and AMR treatment is provided to the allograft recipient; or (ii) the concentration of C4d+/Cd144+ EMP in the blood sample is less that about seven standard deviations above the mean of a reference C4d+/CD144+ EMP concentration from a healthy individual, the allograft transplant recipient does not have AMR and no treatment is necessary.

22. (canceled)

23. (canceled)

24. (canceled)

25. The method according to claim 21, wherein the method further comprises determining donor-specific antibodies in the blood sample from the allograft transplant recipient.

26. The method according to claim 21, wherein the method further comprises obtaining an allograft biopsy.

27. (canceled)

28. The method according to claim 21, wherein if the concentration of CD4+/CD144+ EMP in the blood sample is greater than about eight standard deviations above the mean of the reference C4d+/CD144+ EMP concentration, the allograft recipient has antibody-mediated rejection treatment and AMR treatment is provided to the allograft recipient.

29. The method according to claim 21, wherein if the concentration of CD4+/CD144+ EMP in the blood sample is less than about eight standard deviations above the mean of the reference C4d+/CD144+ EMP concentration, the allograft transplant recipient does not have AMR and no treatment is necessary.

30. A method of identifying an transplant recipient at risk of allograft rejection, wherein the transplant recipient is identified by:
determining the concentration of C4d+/CD144+ EMP in a blood sample obtained from the transplant recipient after transplant and before onset of transplant rejection symptoms;
wherein, the transplant recipient is at risk of allograft rejection if the concentration of C4d+/CD144+ EMP in the blood sample is greater than about seven standard deviations above the mean of a reference C4d+/CD144+ EMP concentration from a healthy individual.

31. (canceled)

32. (canceled)

33. (canceled)

34. The method according to claim 30, wherein the subject exhibits normal serum creatinine levels at the time of sampling.

35. The method according to claim 30, wherein the subject exhibits increased serum creatinine levels at the time of sampling.

36. The method according to claim 30, wherein the method further comprises determining donor-specific antibodies in the blood sample from the allograft transplant recipient.

37. The method according to claim 30, wherein the method further comprises obtaining an allograft biopsy.

38. (canceled)

39. The method according to claim 30, wherein if the concentration of CD4+/CD144+ EMP in the blood sample is greater than about eight standard deviations above the mean of the reference C4d+/CD144+ EMP concentration, the allograft transplant recipient is at risk of allograft rejection.
